(19) United States
(12) Patent Application Publication
(10) Pub. No.: US 2025/0242176 A1
(43) Pub. Date: Jul. 31, 2025

(54) UPDATING RADIOTHERAPY TREATMENT PLANS USING INFORMATION DERIVED FROM WHOLE SLIDE IMAGES (WSIs)

(71) Applicant: Varian Medical Systems, Inc., Palo Alto, CA (US)

(72) Inventors: Svenja Lippok, Uttenreuth (DE); Sushil Beriwal, Wexford, PA (US); Ricky Anupam Sharma, Lightwater (GB); Marta Vilalta, Belmont, CA (US)

(21) Appl. No.: 18/425,921

(22) Filed: Jan. 29, 2024

(51) Int. Cl. A61N 5/10; G16H 50/50

(52) U.S. Cl. CPC A61N 5/1038; A61N 5/1039; G16H 50/50; A61N 2005/1041

(57) ABSTRACT

Systems and methods for adapting treatment plans to the characteristics of individual anatomical pathologies (e.g., tumors), and systems, methods, and devices for implementing an adaptive therapy workflow that automatically updates treatment plans based on whole slide images (WSIs) of the target anatomical pathologies.

### FIELD

The present disclosure relates generally to adaptive radiation therapy, and more particularly, to systems, methods, and devices for adapting treatment plans using information derived from whole slide images (WSIs), and to systems, methods, and devices that enable the adaptation of radiation treatment plans to the characteristics of individual anatomical pathologies as well as anatomical changes in the patient.

### BACKGROUND

Radiation therapy involves medical procedures that use external radiation beams to treat anatomical pathologies (tumors, lesions, vascular malformations, nerve disorders, etc.) by delivering prescribed doses of radiation (X-rays, gamma rays, electrons, protons, and/or ions) to the anatomical pathologies, while minimizing radiation exposure to the surrounding tissue and critical anatomical structures.

In general, radiation therapy treatments consist of several phases. First, a precise three-dimensional (3D) map of the anatomical structures in the area of interest (head, body, etc.) is constructed using any one of (or combinations thereof) a computed tomography (CT), cone-beam computed tomography (CBCT), magnetic resonance imaging (MRI), positron emission tomography (PET), 3D rotational angiography (3DRA), or ultrasound techniques. This determines the exact coordinates of the target within the anatomical structure, namely, locates the tumor or abnormality within the body and defines its exact shape and size. Second, a motion path for the radiation beam is computed to deliver a dose distribution that the radiation oncologist finds acceptable, considering a variety of medical constraints. During this phase, a team of specialists develop a treatment plan using special computer software to optimally irradiate the tumor and minimize dose to the surrounding normal tissue by designing beams of radiation to converge on the target area from different angles and planes. Third, the radiation treatment plan is executed. During this phase, the radiation dose is delivered to the patient according to the prescribed treatment plan. Generally, a treatment plan is delivered to the patient over a series of radiation treatments referred to as fractions.

There are many factors that can contribute to differences between the prescribed radiation dose distribution and the actual dose delivered (i.e., the actual dose delivered to the target during the radiation treatment). One such factor is uncertainty in the patient's position in the radiation therapy system. Other factors involve uncertainty that is introduced by changes that can occur during the patient's treatment. Such changes can include random errors, such as small differences in a patient's setup position. Other sources are attributable to physiological/anatomical changes that might occur if a patient's tumor regresses or if the patient loses weight during therapy. Another category of uncertainty includes motion. Motion can potentially overlap with either of the categories as some motion might be more random and unpredictable, whereas other motion can be more regular.

These changes can cause the target volumes and surrounding anatomical structures and organs to move and change in size and shape during the therapy. As such, continuing to follow the initial treatment plan may result in an actual received dose distribution that differs from the planned dose distribution, and thus reduced doses to target volumes and/or increased doses to organs at risk (OARs). During the treatment delivery phase, therefore, the treatment plan may be adapted to the image of the day to better reflect the current situation.

Adaptive radiation therapy can occur at three different timescales, namely, off-line between treatment fractions, on-line immediately prior to a treatment fraction, and in real-time during a treatment fraction. In an off-line adaptive therapy process, a new image (CT or CBCT image, for example) of the patient is acquired before or after each of the fractions and the images are evaluated to determine multi-day locations of the target volumes. Based on this, a new plan can be developed to better reflect the range of motion of the target volumes. In an on-line adaptive therapy process, the radiation therapy system can be used prior to a fraction to validate or adjust the patient treatment plan for the treatment delivery. The imaging system can thus be used to concurrently modify the treatment delivery to reflect the changes in the patient's anatomy. In a real-time (on-couch) adaptive therapy process, the radiation therapy system can be used during a treatment fraction. On-couch adaptive radiation therapy allows adjustment of the treatment plan based on anatomical changes while the patient is on the treatment table. This may involve performing a fast CBCT scan of the patient prior to treatment delivery, comparing the target volume identified in the CBCT scan (i.e., treatment session target volume) with the target volume identified during the treatment planning phase (i.e., planning target volume) and automatically making modifications to the treatment plan (i.e., adapting the treatment plan) based on the comparison. Since the comparison captures the anatomical changes in the patient, the treatment plan is modified to match the new location and shape of the target volume and surrounding anatomical structures. The planning workflow associated with such modifications allows for the adaptation of the treatment plan to the anatomy of the patient at every fraction while the patient is on the treatment couch.

The above described treatment plan adaptations involve re-scanning the patient prior to the treatment delivery and modifying the treatment plan based on anatomical changes and/or movement of the patient. Since the re-scan of the patient does not capture changes related to the individual characteristics of the tumor itself, such as changes in the susceptibility of the tumor to ionizing radiation, for example, the obtained adapted radiation treatment plans do not account for changes that are specific to the individual tumors. Such changes are, however, important to consider when adapting a treatment plan. For example, the tumor's susceptibility to ionizing radiation may have changed from the initial planning phase and/or between treatment fractions. Changes in the susceptibility of the tumor to ionizing radiation can cause the tumor to react to the ionizing radiation differently from what was initially observed during the treatment planning. For example, the tumor's sensitivity and/or resistance to the ionizing radiation may have increased or decreased since the initial treatment planning or from a previous treatment fraction. As such, continuing to follow the treatment plan may result in the tumor receiving higher or lower doses of radiation than what is required.

It would therefore be beneficial to adapt the treatment plan not only to the anatomical/physiological changes that occur in the patient, but also to the characteristics of the individual tumors.

It would also be beneficial to adapt the radiation treatment plan according to the cellular properties of the tumor.

It would also be beneficial to develop an adaptive radiation therapy workflow where the radiation treatment plan is adapted not only to the anatomical/physiological changes that occur in the patient, but also to the characteristics of the individual tumors.

### SUMMARY

Embodiments of the disclosed subject matter enable adapting a radiation treatment plan according to the cellular properties of the tumor.

In embodiments, the adapting includes obtaining/receiving/accessing a treatment plan or generating a treatment plan based on radiology image data of an anatomy of interest containing a tumor; obtaining a whole slide image (WSI) of the tumor; and updating the treatment plan based on information extracted from the WSI, wherein the extracted information includes information specific to cellular properties of the tumor.

In embodiments, the updating includes using the treatment plan and the WSI as inputs to a plan adaptation module, the plan adaptation module including optimization parameters generated based on the feature vector extracted from the WSI; and generating an updated treatment plan by optimizing the treatment plan based on the average feature vector.

In embodiments, the feature vector includes biomarkers that encode morphological information about the tumor, biomarkers that include information about proliferation status or immune status of the tumor, and biomarkers that indicate the tumor's resistance to ionizing radiation.

Embodiments of the disclosed subject matter also enable an adaptive process that integrates radiation therapy workflow with digital pathology workflow in a seamless manner.

Embodiments of the disclosed subject matter also enable the adaptation of radiation treatment plans to the characteristics of individual anatomical pathologies (e.g., tumors) as well as anatomical changes in the patient.

Embodiments of the disclosed subject matter also allow for a more personalized and better targeted radiography protocol that is adapted to the particular cancer type.

A system configured to perform the treatment adaptation processes disclosed herein is also disclosed.

A system including a computer processing device configured to execute a sequence of programmed instructions embodied on a computer-readable storage medium, the execution thereof causing the system to execute the treatment adaptation protocols disclosed herein is also disclosed.

A non-transitory computer-readable storage medium upon which is embodied a sequence of programmed instructions for the adaptation of treatment plans as disclosed herein, and a computer processing system that executes the sequence of programmed instructions embodied on the computer-readable storage medium are also disclosed. Execution of the sequence of programmed instructions can cause the computer processing system to execute the adaptive workflow described herein.

Objects and advantages of embodiments of the disclosed subject matter will become apparent from the following description when considered in conjunction with the accompanying drawings.

### BRIEF DESCRIPTION OF DRAWINGS

FIG. 1A is a simplified schematic diagram of a radiation therapy system, according to various embodiments of the disclosed subject matter.

FIG. 1B is a simplified illustration for using the radiation therapy system of FIG. 1A for adaptive radiation therapy, according to various embodiments of the disclosed subject matter.

FIG. 2A is a simplified illustration of a treatment plan updating system, according to various embodiments of the disclosed subject matter.

FIG. 2B is a simplified illustration of an adaptive workflow, according to various embodiments of the disclosed subject matter.

FIGS. 3-5 is a simplified illustration of a treatment plan generating system, according to various embodiments of the disclosed subject matter.

FIG. 6 is a simplified illustration of a whole slide image (WSI) analyzer module, according to various embodiments of the disclosed subject matter.

FIGS. 7-8 is a process flow diagram for adapting treatment plans based on information extracted from corresponding WSIs, according to various embodiments of the disclosed subject matter.

FIG. 9 is a simplified illustration of 4D adaptive directives, according to various embodiments of the disclosed subject matter.

FIGS. 10A-10B are simplified illustrations of treatment plan adaptation modules, according to various embodiments of the disclosed subject matter.

FIGS. 11-13 are process flow diagrams for training and applying a deep learning based treatment plan adaptation module, according to various embodiments of the disclosed subject matter.

FIGS. 14-15 are process flow diagrams for adapting treatment plans based on information extracted from corresponding WSIs and shifts in the anatomy of the patient, according to various embodiments of the disclosed subject matter.

FIGS. 16A-16E are process flow diagrams for adapting treatment plans based on information extracted from corresponding WSIs and/or additional data, according to various embodiments of the disclosed subject matter.

FIG. 17 is a process flow diagram for comparing simulations in treatment plans, according to various embodiments of the disclosed subject matter.

### DETAILED DESCRIPTION

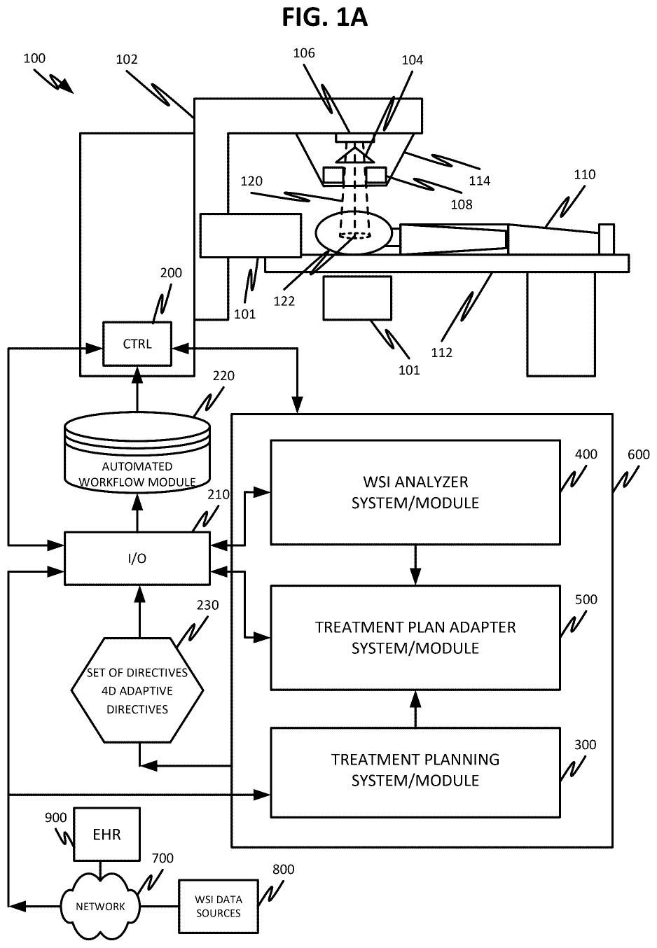

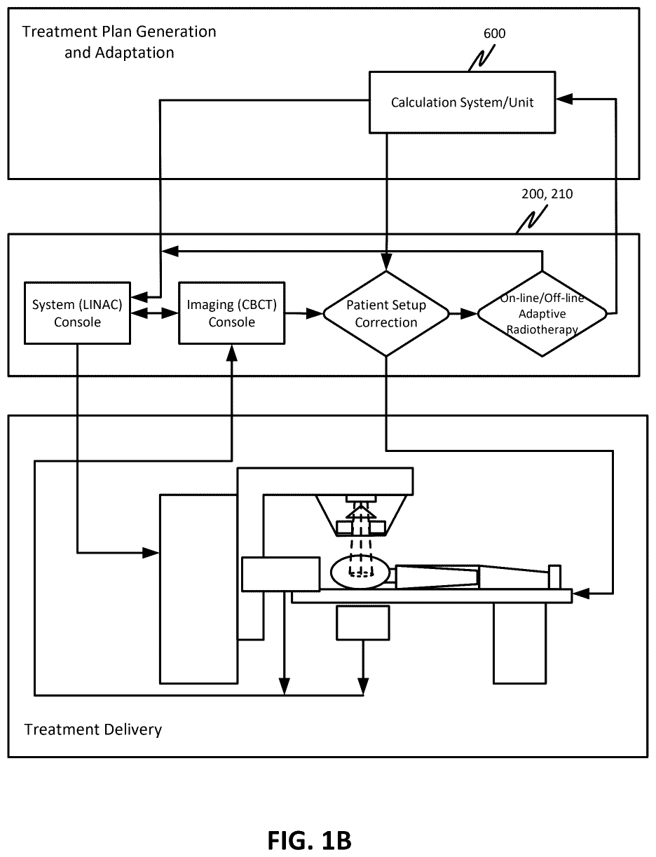

Referring to FIG. 1A, an exemplary radiation therapy system 100 is shown that can be used in adaptive radiation therapy as shown in FIG. 1B. The radiation therapy system 100 can provide radiation to a patient 110 positioned on a treatment couch 112 by implementing the various radiation treatment generating, adapting and verification protocols described herein. The radiation therapy can include photon-based radiation therapy, particle therapy, electron beam therapy, or any other type of treatment therapy.

In an embodiment, the radiation therapy system 100 can include a radiation treatment device 101 such as, but not limited to, a LINAC operable to generate one or more beams of megavolt (MV) X-ray radiation for treatment. The LINAC may also be operable to generate one or more beams of kilovolt (kV) X-ray radiation, for example, for patient imaging. The system 100 has a gantry 102 supporting a radiation treatment head 114 with one or more radiation sources 106 and various beam modulation elements, such as, but not limited to, flattening filter 104 and collimating components 108. The collimating components 108 can include, for example, a multi-leaf collimator (MLC), upper and lower jaws, and/or other collimating elements. The collimating components 108 and/or the flattening filter 104 can be positioned within the radiation beam path by respective actuators (not shown), which can be controlled by controller 200.

The gantry 102 can be a ring gantry (i.e., it extends through a full 360° arc to create a complete ring or circle), but other types of mounting arrangements may also be employed. For example, a static beam, or a C-type, partial ring gantry, or robotic arm can be used. Any other framework capable of positioning the treatment head 114 at various rotational and/or axial positions relative to the patient 110 may also be used.

In an embodiment, the radiation therapy device is a MV energy intensity modulated radiation therapy (IMRT) device. The intensity profiles in such a system are tailored to the treatment requirements of the individual patient. The IMRT fields are delivered with MLC 108, which can be a computer-controlled mechanical beam shaping device attached to the head 114 and includes an assembly of metal fingers or leaves. For each beam direction, the optimized intensity profile is realized by sequential delivery of various subfields with optimized shapes and weights. From one subfield to the next, the leaves may move with the radiation beam on (i.e., dynamic multi-leaf collimation (DMLC)) or with the radiation beam off (i.e., segmented multi-leaf collimation (SMLC)).

Alternatively, or additionally, the radiation therapy device 101 can be a tomotherapy device where intensity modulation is achieved with a binary collimator (not shown) which opens and closes under computer control. As the gantry 102 continuously rotates around the patient 110, the exposure time of a small width of the beam can be adjusted with opening and closing of the binary collimator, allowing radiation 120 to be directed to a portion of the body of the patient 110 and delivered to a region of interest 122 through the most desirable directions and locations of the patient 110. The region of interest is a two-dimensional area and/or a three-dimensional volume that is desired to receive the radiation and it may be referred to as a target or target region or target volume. Another type of region of interest is a region of risk. If a portion includes a region of risk, the radiation is diverted from the region of risk. The patient 110 may have more than one target region that needs to receive radiation therapy.

Alternatively, or additionally, the radiation therapy device can be a helical tomotherapy device, or a simplified intensity modulated arc therapy (SIMAT) device, a volumetric modulated arc therapy (VMAT) device, or a volumetric high-definition (or hyperarc) therapy (HDRT). In effect, any type of IMRT device can be employed as the radiation therapy device 101 of system 100, and can also include an on-board volumetric imaging, which can be used to generate in-treatment image data generated during a treatment session.

For example, embodiments of the disclosed subject matter can be applied to image-guided radiation therapy (IGRT) devices, which uses cross-sectional images of a patient's internal anatomy taken during the radiation therapy treatment session (i.e., in-treatment images) to provide information about the patient's position. Frequent two or three-dimensional imaging during the radiation treatment is used to direct the therapeutic radiation utilizing the imaging coordinates of the actual radiation treatment plan. This ensures that the patient is localized in the radiation treatment system in the same position as planned, and that the patient is properly aligned during the treatment. Although, the IGRT process involves conformal radiation treatment guided by specialized imaging tests taken during the planning phase, it does rely on the imaging modalities from the planning process as the reference coordinates for localizing the patient 110 during treatment. Thus, associated with each image-guided radiation therapy system is an imaging system to provide in-treatment (treatment session) images that are used to set-up the radiation delivery procedure.

In-treatment images can include one or more two or three-dimensional images (typically X-ray) acquired at one or more different points during treatment. There are a variety of ways to acquire in-treatment images. In certain approaches, distinct independent imaging systems and/or imaging methods are used for acquiring pre-treatment and in-treatment images, respectively. For example, a 3D IGRT could include localization of a cone-beam computed tomography (CBCT) dataset with a planning computed tomography (CT) dataset, and a 2D IGRT could include matching planar kilovoltage (kV) radiographs or megavoltage (MV) images with digital reconstructed radiographs (DRRs) obtained from the planning CT.

Alternatively, the system 100 can include a kilovoltage or a megavoltage detector operable to receive the radiation beam 120. The radiation therapy device 101 and the detector can operate as a computed tomography (CT) system to generate CT images of the patient. The images can illustrate the patient's body tissues, organs, bone, soft tissues, blood vessels, etc.

Each type of radiation therapy device can be accompanied by a corresponding radiation plan and radiation delivery procedure.

The controller 200, which can be, but is not limited to, a graphics processing unit (GPU), can include a computer with appropriate hardware such as a processor, and an operating system for running various software programs and/or communication applications. The controller 200 can include software programs that operate to communicate with the radiation therapy device 101, which software programs are operable to receive data from external software programs and hardware. The computer can also include any suitable input/output (I/O) devices 210, which can be adapted to allow communication between controller 200 and a user of the radiation therapy system 100, e.g., medical personnel. For example, the controller 200 can be provided with I/O interfaces, consoles, storage devices, memory, keyboard, mouse, monitor, printers, scanner, as well as a departmental information system (DIS) such as a communication and management interface (DICOM) for storing and transmitting medical imaging information and related data and enabling the integration of medical imaging devices such as scanners, servers, workstations, printers, network hardware, etc.

Alternatively, or additionally, the I/O devices 210 can provide access to a network 700 for transmitting data between controller 200 and remote systems. For example, the controller 200 can be networked via I/O 210 with other computers and radiation therapy systems. The radiation therapy system 100, the radiation treatment device 101, and the controller 200 can also communicate via a network 700 (cloud-based or otherwise) with databases and servers at different locations, such as, hospitals, clinics, field researchers, different databases containing electronic health records (EHRs) 900, and different databases storing whole slide images (WSIs) 800, and/or other databases and servers that provide clinical data, pathology data, omics data, electronic medical image (EMR) data, and any other complementary data including, but not limited to, paraclinical measurements (i.e., blood, urine, lab tests, etc.) and expert conclusions (tumor characterizations, radiologist tumor size measurements, genetic mutations, etc.) relating to the patient and or the tumor, the type or subtype of the cancer, etc. The radiation therapy system 100, the radiation treatment device 101, and the controller 200 can also aggregate the clinical information from these different sources and store it in a digital pathology database, analyze the data by different processing methods, and provide an integrated platform for a fully automated data access, processing, viewing and display.

The radiation therapy system 100, the radiation treatment device 101, and the controller 200 can also communicate with a dose calculation server (e.g., distributed dose calculation framework), and a calculation system 600 for generating and adapting a radiation treatment plan to account for anatomical/physiological changes in the patient as well as for other factors that influence the tumor, including but not limited to the susceptibility of the tumor to ionizing radiation, and other characteristics of the tumor that can be derived from pathology data.

The calculation system 600 may include a treatment planning system/module 300, a whole slide image (WSI) analyzer system/module 400, and a treatment plan adapter system/module 500. Although the calculation system 600 is shown as a separate unit, the calculation system 600 and/or portions thereof may be integrated into the system 100 (e.g., as part of controller 200, or as separate modules within system 100, or integrated into other components of system 100).

The system 100 can also include a plurality of modules containing programmed instructions (e.g., as part of controller 200, or as separate modules within system 100, or integrated into other components of system 100), which instructions cause system 100 to perform different functions related to adaptive radiation therapy or other radiation treatment, as discussed herein, when executed. For example, the system 100 can include a patient positioning module operable to position and align the patient 110 with respect to a desired location, such as the isocenter of the gantry, for a particular radiation therapy treatment, an image acquiring module operable to instruct the radiation therapy system and/or the imaging device to acquire images of the patient 110 prior to the radiation therapy treatment (i.e., pre-treatment/reference images used for treatment planning and patient positioning) and/or during the radiation therapy treatment (i.e., in-treatment session images), and to instruct the radiation therapy system 100 and/or the imaging device 101 or other imaging devices or systems to acquire images of the patient 110.

The system 100 can further include a radiation dose prediction module operable to predict a dose to be delivered to the patient 110 before commencement of the radiation therapy treatment, a dose calculation module operable to calculate the actual dose delivered to the patient 110 during radiation therapy treatment, a treatment delivery module operable to instruct the radiation therapy device 100 to deliver a treatment plan to the patient 110, a correlation module operable to correlate the planning images with the in-treatment images obtained during radiation therapy, a computation module operable to reconstruct three-dimensional target volumes from in-treatment images, an analysis module operable to compute displacement measurements, and a feedback module operable to instruct the controller in real-time to stop radiation therapy based on a comparison of the calculated displacement with a predetermined threshold value (range).

The system 100 can further include one or more contour generation modules operable to generate contours of target volumes and other structures in pre-treatment (planning) and in-treatment (treatment session) images, an image registration module operable to register pre-treatment images with subsequent in-treatment images, a dose calculation module operable to calculate accumulated dose, a contour propagation module operable to propagate a contour from one image to another, a contour verification module operable to verify a generated contour, a registration deformation vector field generation module operable to determine deformation vector fields (DVFs) as a result of an image deformation process. The system 100 can further include modules for electron density map generation, isodose distribution generation, does volume histogram (DVH) generation, image synchronization, image display, treatment plan generation, treatment plan optimization, automatic optimization parameter generation, updating and selection, and adaptive directives and treatment information transfer. The modules can be written in the C or C++ programming language, for example. Computer program code for carrying out operations as described herein may be written in any programming language, for example, C or C++ programming language.

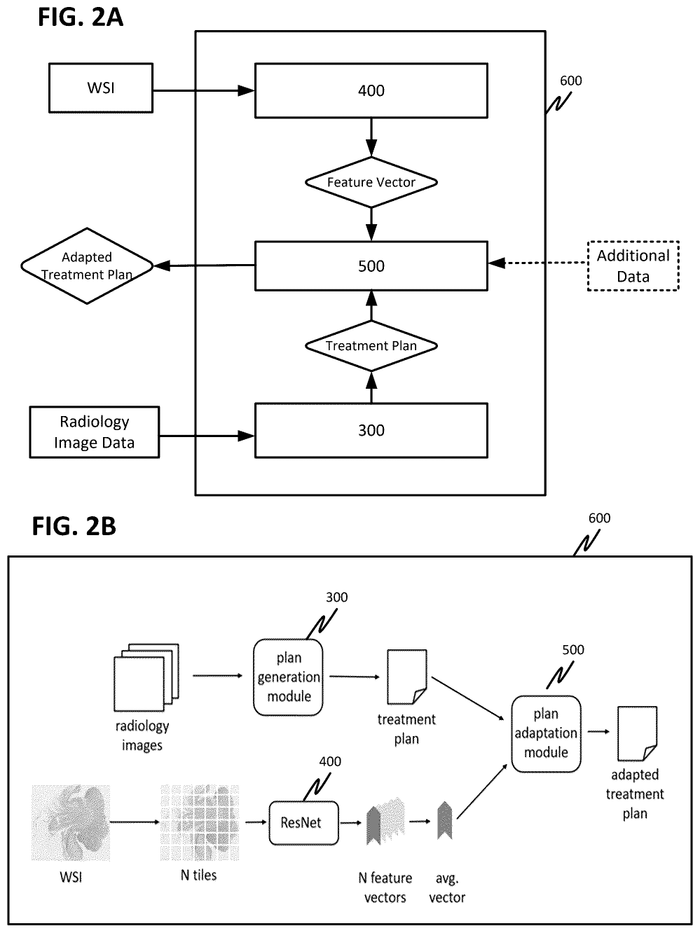

As shown in FIGS. 1A, 1B, 2A and 2B, the calculation system 600 may include a treatment planning system/module 300 configured to generate a treatment plan based on radiology image data, a WSI analyzer system/module 400 configured to extract feature vectors from a WSI image, and a treatment plan adapter system/module 500 configured to update (adapt) the treatment plan generated by the treatment planning system/module 300 based on the extracted feature vector, and optionally, based on additional radiology and/or pathology data. By taking WSIs and other pathology data into account, a radiotherapy treatment plan can be further adapted according to the cellular properties of the tumor, which allows for more personalized and better targeted radiotherapy protocol that is adapted for the particular cancer type.

Alternatively, the treatment planning system/module 300 is not part of the calculation system 600. In such a case, the treatment plan is generated external to the calculation system 600, and the calculation system 600 is configured to receive and/or obtain and/or access the treatment plan from the treatment planning system/module 300. Alternatively, the calculation system 600 may receive/obtain/access a previously generated treatment plan and associated treatment parameters/instructions/directives from a database/server where the treatment plan and associated treatment parameters/instructions/directives have been stored. Such database/server may be external to the radiation therapy system 100.

The general workflow for adapting a treatment plan according to the cellular properties of the tumor is shown in FIG. 2B, and it may involve a first step in which a radiotherapy treatment plan is created, which may involve obtaining radiology image data of the patient's body part to be treated, determining a treatment isocenter and inputting both in a treatment plan generation algorithm, a second step in which a whole slide image (WSI) of a specimen of the cancerous tissue to be treated is obtained, which may involve querying a digital pathology database for any existing WSIs and/or ordering the preparation of a WSI (including the indication of biopsy coordinates, staining protocols, etc.), a third step in which the WSI and the radiotherapy treatment plan are input into a treatment plan update algorithm which has been trained to update radiotherapy treatment plans by automatically analyzing corresponding WSIs, and a fourth step in which the updated radiotherapy treatment plan is provided either to a user for further evaluation or the radiotherapy treatment system 100 for execution.

Alternatively, the general workflow for adapting a treatment plan according to the cellular properties of the tumor may involve a first step in which a previously generated radiotherapy treatment plan is obtained/received/accessed, which may involve communicating with the treatment planning system/module 300 and/or the external database/server to access the treatment plan, a second step in which a whole slide image (WSI) of a specimen of the cancerous tissue to be treated is obtained, which may involve querying a digital pathology database for any existing WSIs and/or ordering the preparation of a WSI (including the indication of biopsy coordinates, staining protocols, etc.), a third step in which the WSI and the radiotherapy treatment plan are input into a treatment plan update algorithm which has been trained to update radiotherapy treatment plans by automatically analyzing corresponding WSIs, and a fourth step in which the updated radiotherapy treatment plan is provided either to a user for further evaluation or the radiotherapy treatment system 100 for execution.

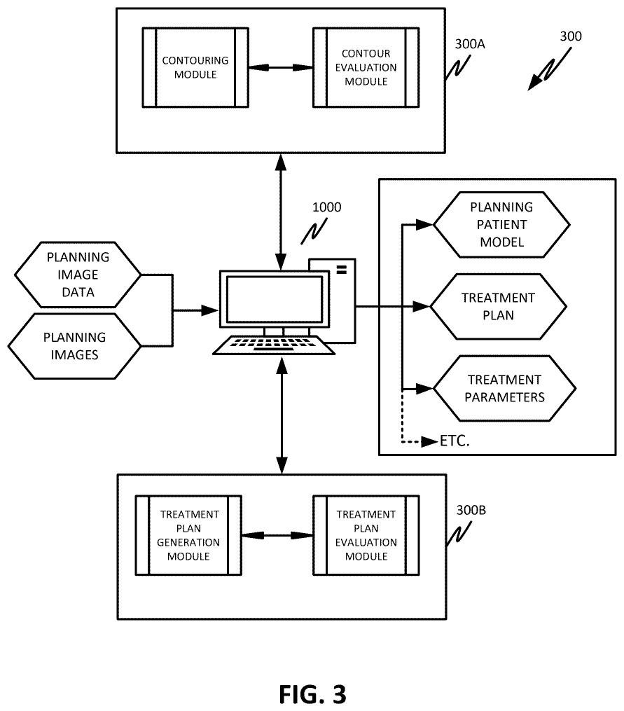

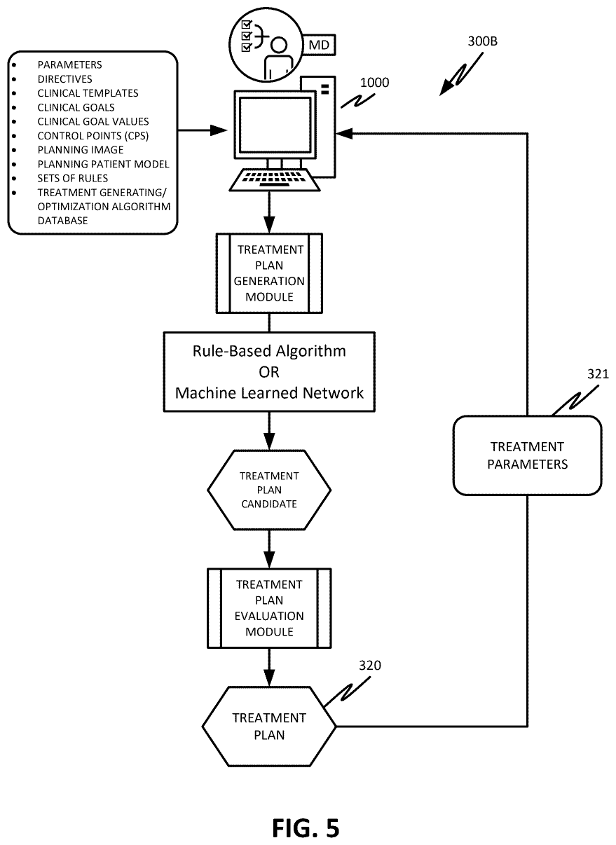

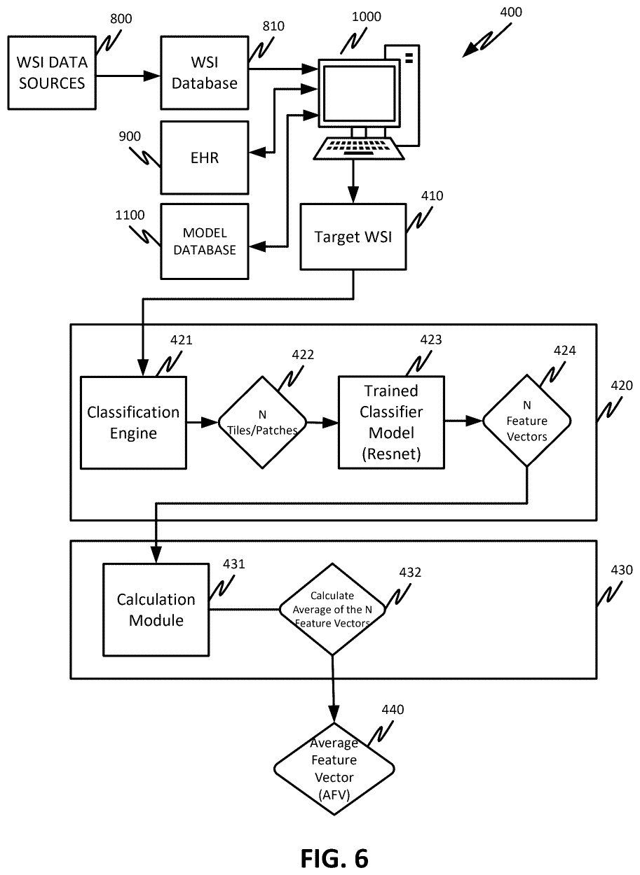

The treatment plan may be a treatment plan generated at the initial planning phase and/or a treatment plan that is an adaptation of an earlier treatment plan (i.e., a treatment plan adapted for a previous/different treatment session, for example). An exemplary treatment plan generating system 300 that can be used to generate a treatment plan is shown in FIGS. 3-5. In a typical planning process, qualified medical personnel (physician) manually draws contours on one or more planning images, such as CT images, for example (i.e., radiology images). These contours delineate the malignant tumor (target) that is to be irradiated, as well as one or more other structures, such as organs, tissue, etc. that are susceptible to substantial damage from radiation exposure (OARs). The planning radiology images can also be semi-automatically or automatically delineated using a contouring and evaluation module 300A of a computer processing system 1000 that may be part of the calculation system 600 or part of the controller 200. The accepted target contours 311, 312 on the planning image (i.e., CT image) represents the planning patient model 310.

To generate a treatment plan, the medical personnel (physician) generates a list of treatment parameters, such as but not limited to, the targets for which the radiation is to be maximized, target for which the radiation is to be minimized, and other parameters and directives related to the specific radiation system 100 that will deliver the treatment plan to the patient 110. The physician also specifies a preferred dose distribution for the target structures. The dose distribution may be expressed as a set or a template of clinical goals (CG), which are suitable goals of radiation doses for the treatment of the patient. These clinical goals (CG) can be given for example in the form of mean dose of radiation (in Gray) to a target structure and the dose that certain volume of an organ, such as an organ at risk (OAR), must not exceed.

Clinical goals, however, may also be given in other dimensions that are not in the form of dose of radiation to a target structure and dose to volume of organ. Each of the given goals can further be ordered in priority describing the importance of meeting a goal in comparison to another goal. Such a set is referred to as a prioritized set of clinical goals (prioritized CG). Each clinical goal can be expressed as a quality metric Q and its associated goal value. An exemplary prioritized set of clinical goals is:
GOAL 1: Target (PTV) must receive 50Gy: Priority 1
GOAL 2: Organ at risk X (OARx) must receive less than 25 Gy: Priority 2
GOAL 3: Organ at risk Y (OARy) must receive a mean dose of less than 30 Gy: Priority 3

Using one of a rule-based algorithm or a machine learned network, the treatment plan generating module 300B then automatically generates an optimized plan for the planning patient model 310 by minimizing, via an optimization process, a cost function defining the dose distribution for the set of prioritized clinical goals (CG). There are many algorithms that can be applied to minimize a cost function, including but not limited to, calculating the gradient of the cost function. The solution to the optimization process results in treatment parameters being determined for the treatment plan candidate. To optimize the treatment plan, at the outset of the treatment planning process, a number of control points (CPs) are also specified for the beam trajectory that takes into consideration the beam shaping elements of the radiation therapy system 100. Each control point (CP) is associated with a set of treatment parameters, including but not limited to, a set of (MLC) leaf positions, (MLC) shape, gantry rotation speed, gantry position, dose rate, and/or any other parameters. The number and position of the control points (CPs) may be set in any convenient manner, such as, but not limited to, by using the treatment planning software, or by the system operator. Based on the treatment parameters, a dose distribution within the treatment volume can be calculated for each control point (CP) by any number of techniques, such as, but not limited to, pencil beam convolution, or any other suitable algorithm, and the radiation dose distribution for each (CP) can be associated with the corresponding gantry angle, (MLC) configuration, and monitor unit (MU). As such, during treatment delivery, the extracted (CP) parameters can be associated with corresponding calculated dose distributions for each (CP).

The physician can also evaluate the treatment plan candidate. The accepted treatment plan candidate becomes the treatment plan 320. The treatment plan 320 and associated parameters 321 (i.e., clinical goals, dose specification, clinical goal values, treatment plan 3D dose, how the plan was optimized, control points, etc.) are stored in a storage device of the treatment planning system 300 or a storage device of the computer processing system 1000, to be later retrieved by the radiation therapy system 100 for operating the system 100 to deliver radiation treatment according to a chosen radiation treatment plan.

A rule-based algorithm as described in US Publication No. 2019/0299025 A1, and a machine learned network as described in US Publication No. 2020/0121951 A1, both incorporated by reference herein in their entireties, can be used to generate the treatment plan 320 and corresponding treatment parameters 321.

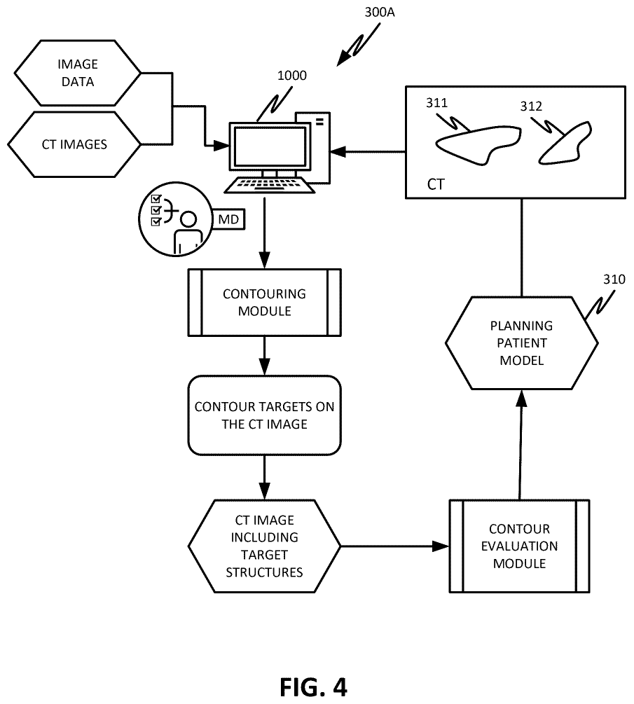

FIG. 4 shows an exemplary WSI analyzer system/module 400 configured to extract feature vectors (i.e., tissue and/or cell morphology classifications or regressions) from a whole slide image (WSI) 410 of the patient 110. Whole slide images (WSIs) represent a dataset of medical images obtained by scanning specimens on glass slides and transforming them into digital files. WSIs are digitized representations of thin sections of stained tissue from various patient sources, such as, biopsy, resection, exfoliation, and fluid. WSIs contain complete histological sections which enable pathologists to see individual cells and subcellular structures, and extract genetic, molecular, and histopathological information therefrom. The WSIs may be stored in a digital pathology database that can be accessed, analyzed and shared among scientists, oncologists, and other professionals within the field to prescribe appropriate cancer treatments.

The target WSI 410 is the digital image of a specimen of the cancerous tissue to be treated in the patient 110. The target WSI 410 may be obtained from the digital pathology database 810, for example, if previously prepared for the patient 110. Otherwise, an order may need to be sent out to a hospital, clinic, pathology laboratory, etc., for the preparation of the target WSI 410. The order may include specific requests for WSI preparation, including, but not limited to specific biopsy coordinates, staining protocols, specific image analysis algorithms, etc.

The computer processing system 1000 uses a trained machine learning architecture 420 including a classification engine 421 to generate a grid comprising of N number of tiles/patches 422, each of the N tiles/patches 422 corresponding to a descriptive vector, and a trained classifier model 423 trained to determine a WSI-level tissue and/or cell morphology classification or regression (i.e., feature vector) 424 for each of the corresponding N tiles/patches. Various machine learning architectures may be used to generate the feature vectors, including, but not limited to, Inception-v3, resnet34, resnet50, resnet152, densenet169, densenet201, or other deep learning or machine learning architectures, such as Fully Convolutional Networks (FCNs), Convolutional Neural Networks (CNNs), or an ensemble thereof. The trained classification models 423 may be selected from a model database 1100 for execution by the computer processing system 1000.

WSI-level tissue and/or cell morphology classifications or regressions (i.e., feature vectors) may include, but are not limited to, visual biomarkers that may encode morphological information such as the shape characteristics of tumor cells or tumor infiltration, specific markers based on specific stains, such as those used for information on proliferation status or immune status, type/subtype of cancer, grade of cancer, percentage of true lymphocites, percentage of tumor-infiltrating lymphocites, RNA expressions, mutation burden, allele frequency, etc.

An exemplary method for extracting feature vectors using trained machine learning classification architectures is disclosed in US Publication No. 2022/0180626 A1, which is incorporated herein by reference in its entirety.

To calculate an average feature vector (AFV) 440, a calculation system 430 is used including a calculation module 431 that calculates 432 the average of the N extracted feature vectors 424.

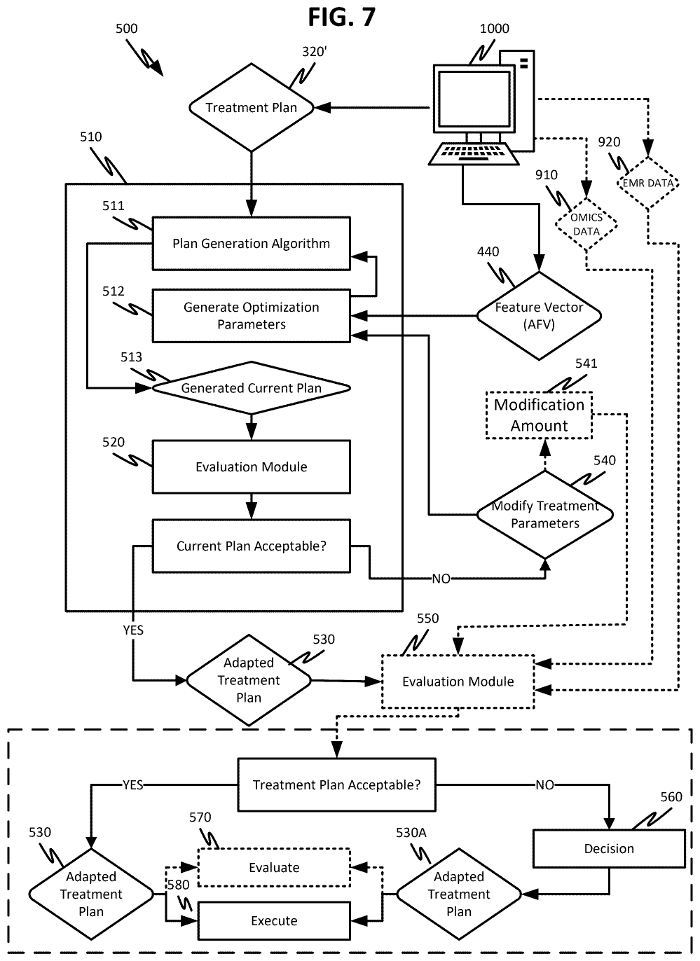

An exemplary treatment plan adapter system/module 500 is shown in FIG. 7. The treatment plan adapter system/module 500 includes an adapted treatment plan generation module 510, an evaluation module 550, and a decision making module 560.

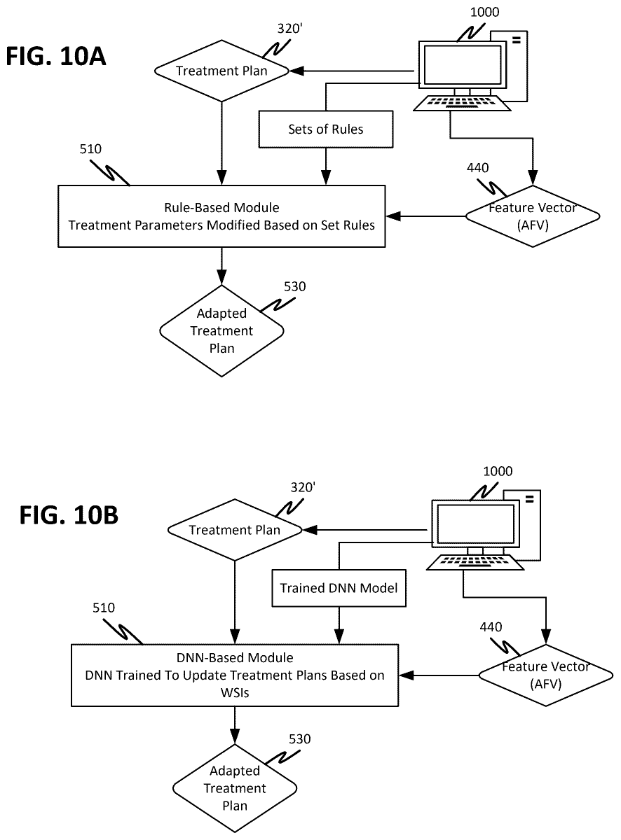

The treatment plan generation module 510 can be a rule-based module (FIG. 10A) or a deep-learning-based module (FIG. 10B) and includes a plan generation algorithm 511 that takes a treatment plan 320′ and the extracted feature vector 440 as inputs to generate a current plan 513 which is optimized using optimization parameters 512. The optimization parameters 512 are determined based on the feature vector 440. These optimization parameters 512 are used to obtain a new treatment plan, namely, an adapted treatment plan 530, which meets the original clinical goals to a similar degree as the treatment plan 320′.

In an exemplary embodiment, the treatment plan 320′ is the treatment plan 320 generated by the treatment plan generating system 300, which may be a treatment plan generated in the planning phase or during a previous treatment session.

Alternatively, the treatment plan 320′ is a previously generated treatment plan for the patient 110 that is accessible by the calculation system 600.

Alternatively, the treatment plan 320′ is a treatment plan that is an adaptation of an earlier treatment plan (i.e., a treatment plan adapted for a previous/different treatment session, for example) and saved in the database/server to be received/obtained/accessed by the calculation system 600.

The optimization parameters 512, such as the radiation/dose parameters for example, may be used to re-calculate the radiotherapy treatment plan 320′ using the treatment plan generation algorithm 511. The optimization parameters 512, in turn, may be determined based on a visual biomarker 440 extracted from the target WSI 410. The visual biomarker may be standardized across tumors so as to indicate the resistance of the respective tumor type against ionizing radiation. Further, the biomarker may encode morphological information, e.g., shape characteristics of tumor cells or tumor infiltration, or be specific markers based on specific stains, e.g., used for information on proliferation status or immune status, for example. The listed biomarkers are exemplary only and any other molecular, physiologic, histologic, and radiographic biomarkers that are extracted from the WSI may be used to generate the optimization parameters 512 to recalculate the radiation/radiation dose parameters of the treatment plan.

In a rule-based adaptation module 510, the treatment parameters 540 may be deterministically modified based on the presence of certain readouts in the feature vector 440. For example, the radiation dose or radiation dose rate may be increased or decreased based on certain readouts, such as proliferation levels, the % of necrotic cells, a susceptibility to ionizing radiation, etc., in the feature vector 440. The set of rules may be a previously determined set of rules, which can be stored in a memory of the computer processing system 1000 and accessed when the adaptation algorithm is executed.

In a deep learning-based module 510, a trained deep neural network (DNN) is employed which is trained to update the treatment parameters 540 based on the readout from the target WSI, as shown in FIG. 13. The DNN refers to a class of computer-based machine-learning algorithms that utilize many layers or stages (in particular, at least two “hidden” layers between input and output layers) of data processing for feature learning, pattern analysis, and/or classification. In general, these DNN models are formed by a layered network of processing elements (referred to as neurons or nodes) that are interconnected by connections (referred to as synapses or weights). The layers of nodes are trained from end-to-end (i.e., from input layer to output layer) to extract feature(s) from the input and classify the feature(s) to produce an output (e.g., classification label or class).

Each hidden layer has respective nodes, which perform a particular computation and are interconnected to nodes in adjacent layers. For example, each node can include a weighting function, which provides weights to respective inputs, and an activation function, which processes the weighted inputs to generate respective outputs. The different hidden layers can include, but are not limited to, final loss layers, non-linear operator layers, pooling layers, subsampling layers, fully connected layers, and convolutional layers.

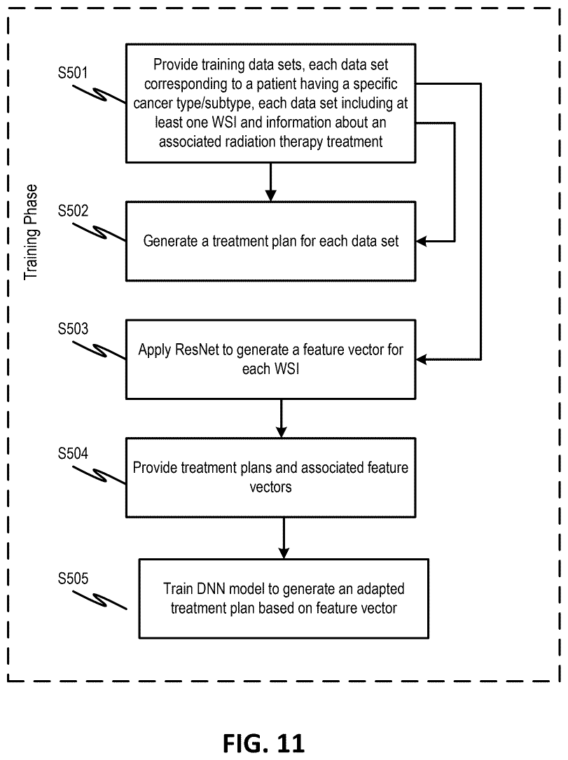

In general, operation of each DNN model involves a training phase and an inference phase. In the training phase, the DNN model uses training data sets to generate a particular output. For example, as shown in FIG. 11, the training data set S501 can include data sets of a plurality of patients (>1000) suffering from a particular cancer type/subtype. The data set of each patient may respectively comprise at least one WSI taken prior to radiation therapy treatment. The data set of each patient may also include information about a (successful) radiation therapy that followed as the ground truth information. Optionally, the data set may additionally comprise radiology image data taken before the radiotherapy. The data sets may be data sets stored at remote servers and accessed via the network 700.

As used herein, “training” refers to determining one or more parameters of nodes in hidden layers of the DNN model, for example, by an iterative process that varies parameters such that the DNN model output more closely matches corresponding ground truth. For example, nodes in the hidden layer can include a filter or kernel, parameters of which (e.g., kernel weight, size, shape, or structure) can be adjusted during the training process.

In training phase, the process can proceed to S502, where a treatment plan is generated for each training data set S501. The generating of the treatment plans may use any of the treatment plan generation methods described herein. Alternatively, each treatment plan may be a default treatment plan which follows specific guidelines for each case. Alternatively, each treatment plan may be generated by applying a known and verified treatment plan generation algorithm. In S503, a trained classification algorithm, such as a ResNet50 is applied to the WSIs of the training data set to generate corresponding feature vectors. The treatment plans generated in S502 and the corresponding feature vectors extracted from the WSIs in S504 are next input into the DNN model in S505 to train the DNN model to generate an adapted treatment plan based on feature vectors.

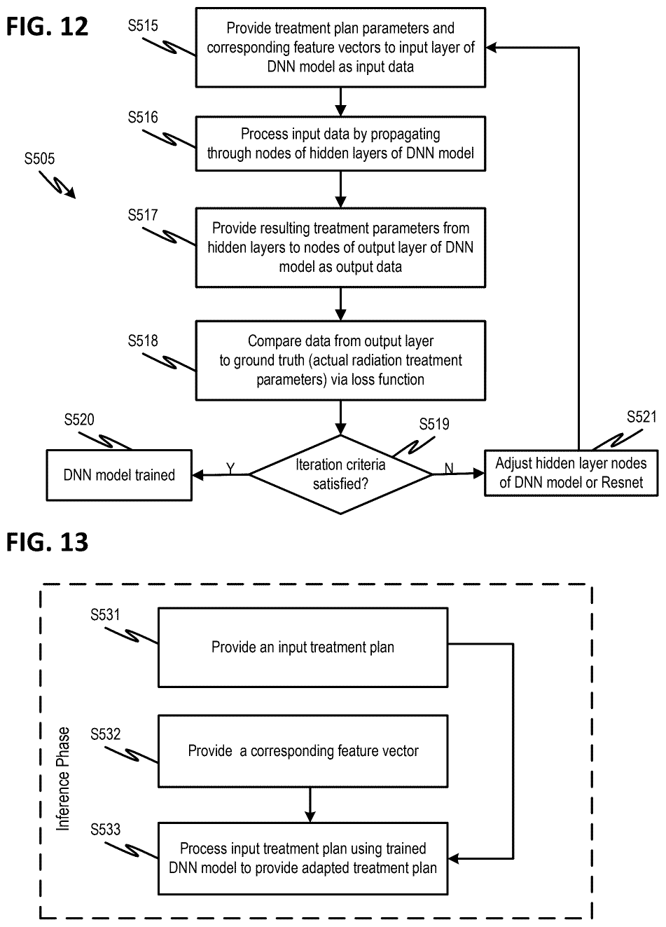

The training of the DNN model can follow a process flow as shown in FIG. 12. In particular, at S515, the parameters of the treatment plans generated in S502 and corresponding feature vectors obtained in S504 are input to the DNN model, for example, to its input layer. At S516, the DNN model processes the input data by propagating through nodes of its hidden layers. At S517, the DNN model produces an output (i.e., treatment parameters as output data) by providing the resulting treatment parameters from hidden layers to nodes of the output layer of the DNN model. The output at S517 can be compared to the ground truth, namely, the parameters of the actual treatment plan applied in the radiation therapy via a loss function at S518. For example, loss function can be mean-squared error, dice loss, cross entropy-based losses or any other loss function known in the art.

During the training, the DNN model is given feedback (by loss function) on how well its output S517 matches the correct output (i.e., ground truth parameters). Once an iteration criteria is satisfied at S519 (e.g., loss function meets a predetermined threshold, a threshold number of iterations has been reached, or no further improvement is seen between iterations), the DNN model is fixed at S520. Otherwise, the training proceeds to S521, where the DNN model is modified by adjusting either the parameters of the hidden layer nodes of the DNN model itself or the hidden layers of the ResNet in order to improve the match between the output at S517 and the desired output. The training process can iterate repeatedly until the desired iteration criteria is met at S519.

In some embodiments, the training data set can include additional subsets. For example, the data set can include a validation set that is used to track the quality of the DNN model during training thereof (e.g., at S519 in FIG. 12), but is not otherwise used as input to the DNN model during training. Alternatively, or additionally, the training data set can include a test subset that is only used after training to quantify the quality of the trained DNN model (e.g., accuracy, dice score) and/or to verify that the DNN model has not over-learned or under-learned the data.

During the inference phase, the trained DNN model S520 operates on an input treatment plan S531 to provide an adapted treatment plan at S533 based on input corresponding feature vectors at S532, as shown in FIG. 13. As such, when the treatment plan 320′ and the corresponding feature vector 440 is input to the trained DNN model S520 of the deep learning-based module 510, the DNN model outputs a corresponding adapted treatment plan (i.e., parameters of the output treatment plan).

Each respective DNN model may run on a corresponding DNN engine, which refers to any suitable hardware and/or software component(s) of a computer system that is capable of executing algorithms according to any suitable deep learning model. In embodiments, the deep learning model(s) can be based on any existing or later-developed neural network, or combinations thereof. Exemplary neural networks include, but are not limited to, a convolutional neural network (ConvNet or CNN) (e.g., U-Net, deep CNN, LeNet, V-Net, AlexNet, VGGNet, Xception, DenseNet, GoogLeNet/Inception, etc.), residual neural network (ResNet), recurrent neural network (RNN) (e.g., Hopfield, Echo state, independent RNN, etc.), long short-term memory (LSTM) neural network, recursive neural network, generative adversarial neural networks (GANs), and deep belief network (DBN).

The adapted treatment plan 530 obtained by executing either the rule-based or the deep learning-based module 510 in FIG. 7 may be accepted as the adapted treatment plan to be executed by the radiation therapy system 100 to irradiate the patient 110.

Optionally, the adapted treatment plan 530 may be further evaluated in the evaluation module 550 before it is delivered to the patient 110. The evaluation may be based on additional information/data regarding the patient and/or the amount of adjustments made to the treatment parameters in 540.

For example, the evaluation module 550 may compare the optimized radiation dose (i.e., the radiation dose required by the adapted treatment plan 530) to the radiation dose required by the treatment plan 320′. If the difference is more than an accepted amount (i.e., the modification amount in 541 is higher than an accepted threshold and thus the optimized radiation dose requires too high of an escalation), the adapted treatment plan 530 may be determined as not appropriate for delivery.

Alternatively, or additionally, the adapted radiation treatment plan 530 may also be evaluated based on additional omics data 910 and/or EMR data 920 pulled from the HER storage 900. For example, omics data, namely, data obtained by measuring biological molecules in the field proteomics, transcriptomics, genomics, metabolomics, lipidomics, epigenomics, and radiomics, such as, but not limited to, global analyses of proteins, RNA, genes, metabolites, lipids, and methylated DNA or modified histone proteins in chromosomes, for example, may be used to evaluate the adapted radiation treatment plan 530. Additional EMR data, such as, but not limited to, electronic medical records indicating medical and treatment history of the patient 110, and or additional data such as, but not limited to, demographic data, previous diagnoses, reports, comorbidities, dose limits, etc. that can be pulled from the EHR storage 900 may also be used to evaluate the adapted treatment plan 530.

Additionally, or alternatively, in order to extract information from the omics data 910 and the EMR data 920 that is relevant to the patient 110, a neural network (NN), such as a large language model (LLM) may be applied on the large data set.

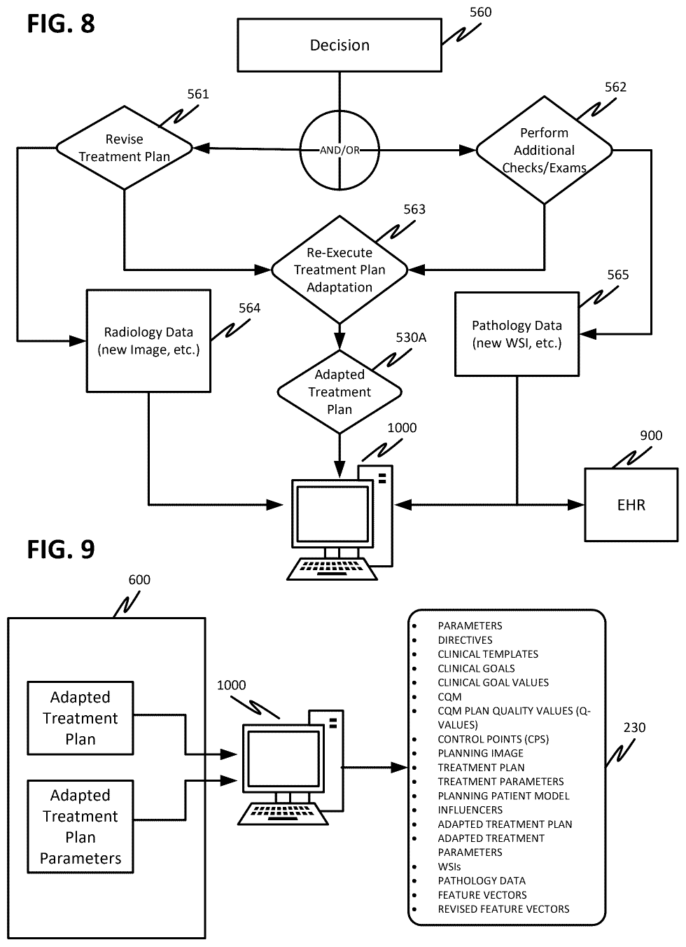

The evaluation may be based on applying an omics-based prediction model trained to predict adaptive radiation therapy eligibility in patients with a particular cancer, for example. By applying an omics-based prediction model on the omics and EMR data set, a prediction can be made as to the efficacy of the adapted treatment plan 530 on the tumor. For example, by applying a trained omics-based prediction model, a prediction could be made as to the tumor shrinkage, and/or treatment perturbations, and/or treatment aggressiveness, and/or geometric and morphologic condition of patient anatomy if the adapted radiation treatment plan 530 is applied. Based on the result of that prediction, the adapted radiation treatment plan 530 may be accepted as the radiation treatment plan to be delivered to the patient 110 or a further decision could be made in 560 as shown in FIG. 8.

Based on the outcome of the evaluation in 550, a decision can be made in 560 to either revise (561) the treatment plan 320′ and re-execute (563) the treatment plan adaptation process in 510, and/or perform additional checks/exams in 562. This may involve ordering new comprehensive radiology imaging exams, new biopsies, the preparation of additional WSI, lab tests, etc. Further, additional, already existing WSIs (different to the one analyzed) may be included and analyzed to safeguard the result.

Additionally, or alternatively, alternative or accompanying therapies may be suggested based on the available information and the results of the evaluation. These alternative therapies may comprise chemotherapy, immunotherapy, surgery, etc. Additional exams may also be performed such as lab tests, sequencing, additional radiology imaging exams, additional digital pathology exams, etc., in order to support such a decision. The newly acquired WSI for the patient may be used to recalculate the feature vectors and to re-execute in 563 the treatment plan adaptation process in 510.

As shown in FIG. 9, the physician also develops a set of adaptive directives 230, which is a list of parameters/directives/information that describes the intent of the adaptive treatment, namely, the 4D description of the planned treatment for the patient 110. The set of adaptive directives can include information regarding the planned dose specification (i.e., Rx prescription), whether adaptive or standard IGRT therapy is to be used, the prescribed clinical goals, such as but not limited to, the target dose coverage and (OAR) risk dose limits, planned clinical goal values, the planning image, supporting images with their corresponding registration information (PET, MRI, etc.), the planned patient model (i.e., the contours of the target structures, such as the target volumes, OARs and other internal dose derived structures on the planned image), the treatment plan (RT Plan), the treatment plan 3D dose (i.e., RT 3D dose), a list of the target structures (target volumes, OARs, influencer structures, body outlines), a list of influencer structures of different treatment sites, information regarding the shapes and location of the planned structures on the planned image, WSIs, pathology data, feature vectors, as well as any information as to how the treatment plan was optimized.

The radiology data 564 of the revised treatment plan 561 as well as the new WSIs and accompanying digital pathology data obtained by performing new tests, exams, etc. in 562 may also be stored in the computer processing system 1000 and/or transferred into the EHR database 900.

The adapted radiation treatment plan 530 may further be evaluated in 570 based on a determination as to the amount of shift in the anatomy of the patient 110 between when the treatment plan 320′ was generated and when the patient 110 is ready for the first and/or a subsequent treatment session in the radiation therapy system 100.

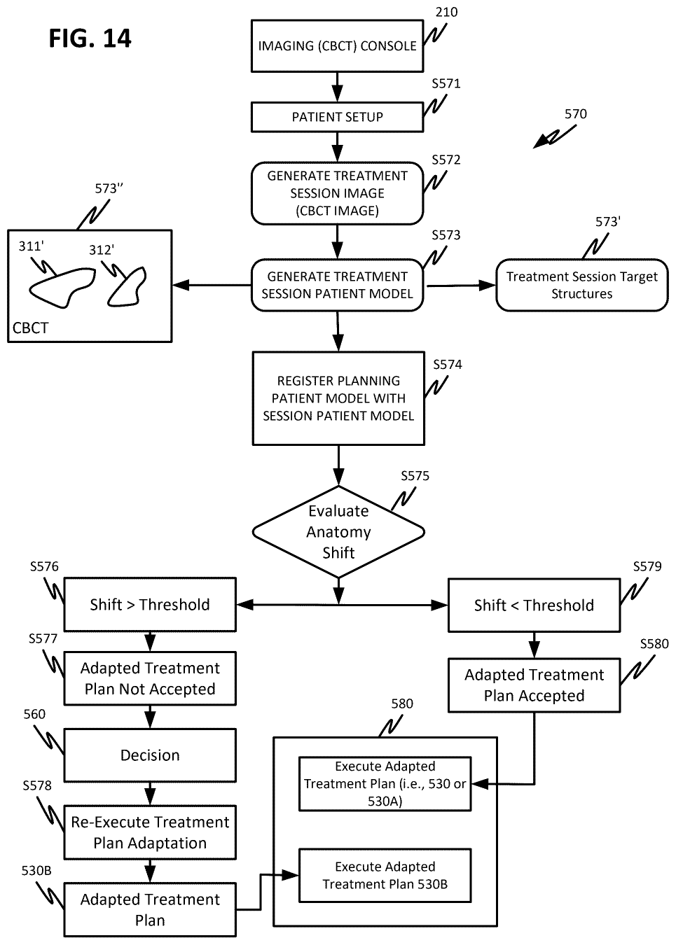

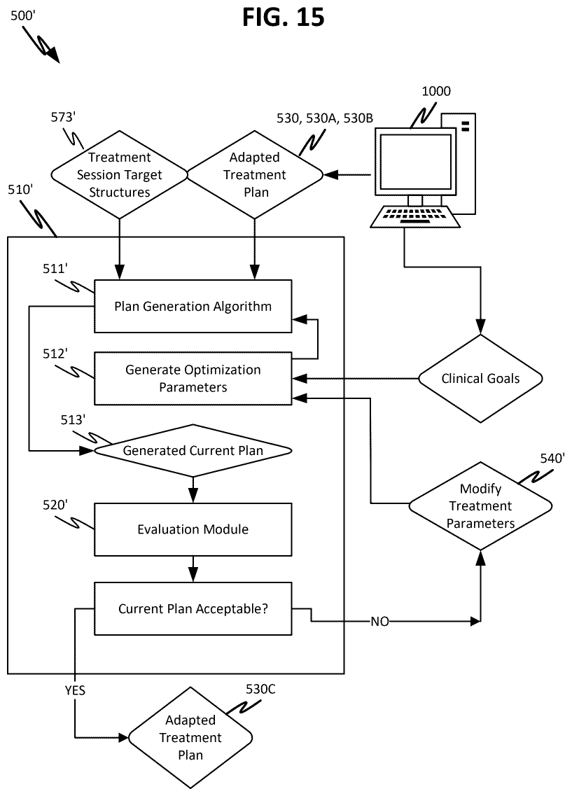

As shown in FIG. 14, to evaluate the shift in the anatomy, first the patient 110 is positioned on the treatment couch 112 and moved to the imaging position (S571) using the imaging console 210. After the patient setup, the next step is to acquire, prior to the treatment, at the treatment site, one or more treatment session images S572 of the portion of the patient 110 that is of interest, using the radiation imaging device 101. In an exemplary embodiment, the treatment session image is a 3D or 4D CBCT scan for example obtained prior to a treatment session by irradiating the region of interest of the patient 110 with radiation 120. This treatment session image (i.e., CBCT image) may show boney structures of the patient 110 but does not include any delineations of target volumes or other structures. Then, in S573 contours of the target structures (S573′) are generated on the treatment session image (CBCT image) to obtain a treatment session patient model 573″. The treatment session patient model 573″ is the CBCT image with the contoured targets 311′, 312′.

The treatment session patient model (i.e., the CBCT image with the contoured targets) 573″ is then compared with the planning patient model 310 (i.e., CT image with contoured targets 311, 312) in S574 to determine the difference between the targets in the planning image and those in the CBCT. This can be done by overlaying the two images and using information such as the shapes and positions of the targets 311, 312 in the planning patient model 370 to calculate the positions, shapes and locations of the target structures 311′, 312′ on the treatment session image (CBCT image). Based on that calculation, a determination can be made in S575 as to whether the target structures represent the same anatomical regions of the patient 110 as those represented by the target structures in the planning image and whether the difference in the shape/position/location of the target structures in the two images are different. The difference in S575 represents a shift in the target anatomy. An exemplary generation of the treatment session patient model and the determination of patient anatomy shift is described in US Publication No. 2020/0121951 A1, which is incorporated by reference herein in its entirety.

If it is determined in S579 that the shift in the target anatomy is below a predetermined threshold, the adapted treatment plan (530 or 530A) may be accepted in S580 to be executed in 580.

On the other hand, if the anatomy shift is above a predetermined threshold (S576), the adapted treatment plan (530 or 530A) is not accepted in S577 since the treatment plan 320′ is no longer acceptable. As such, a decision can be made in 560 to revise the treatment plan 320′ and/or order additional tests, exams etc., to determine if the tumor morphology has been changed. The treatment plan adaptation process is then re-executed in S578 to generate an adapted treatment plan 530B. The adapted treatment plan 530B may then be executed in 580.

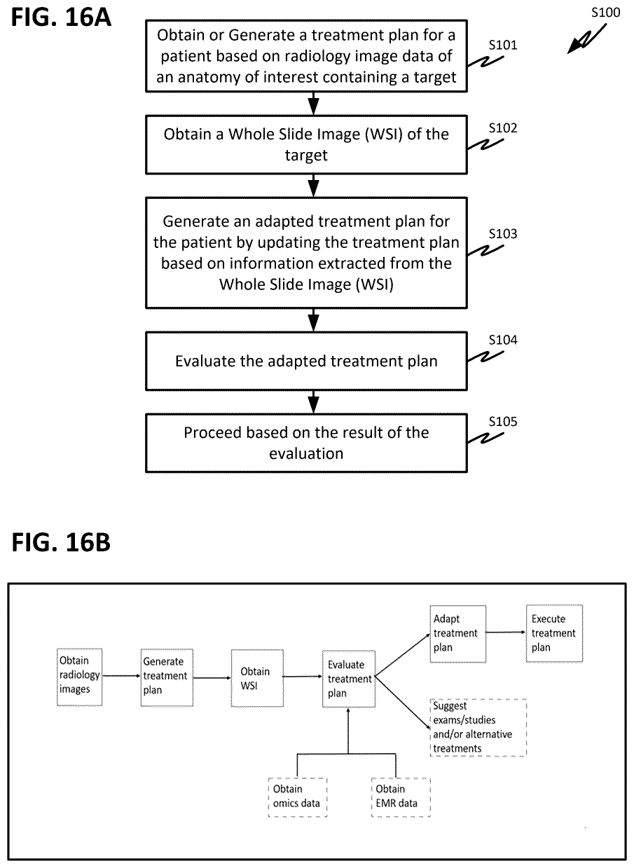

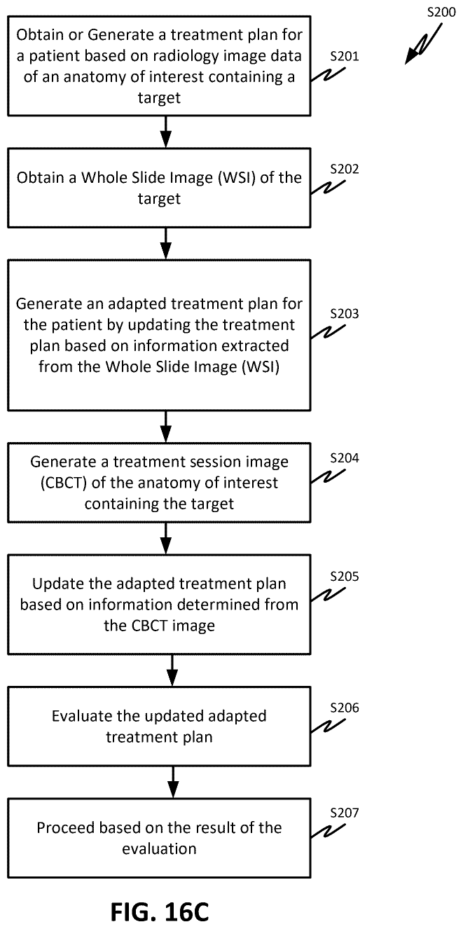

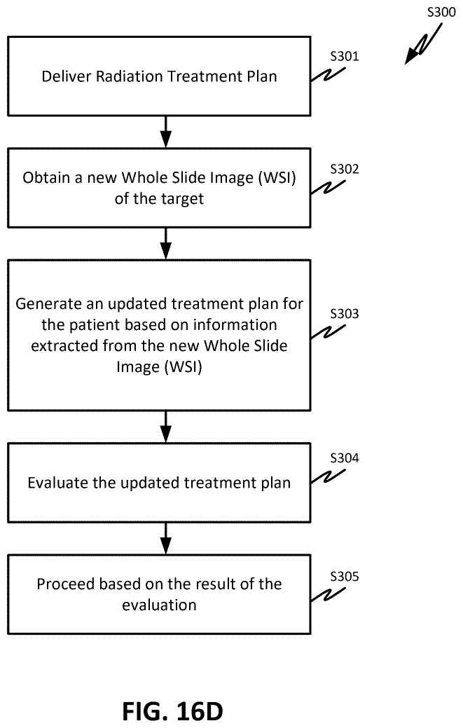

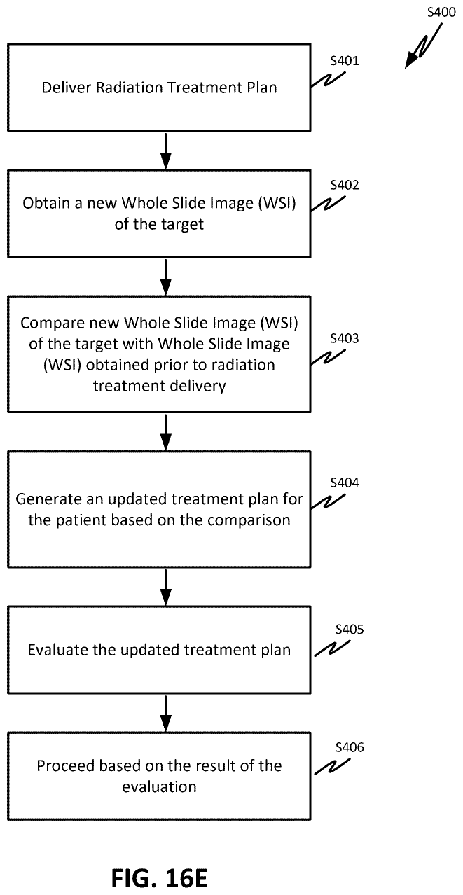

The adapted treatment plan (530, 530A, 530B) may additionally be revised based on the change in the anatomy of the patient as shown in FIG. 16. In order to adapt the treatment plan to the changes in the anatomy of the patient, the treatment session target structures 573′ and the adapted treatment plan (530 or 530A or 530B) are used as inputs to an automated plan generation algorithm in 511′. The plan generation algorithm combines several components from existing components (Photon Optimization algorithm (PO-GPU) for VMAT and IMRT, SmartLMC algorithm for leaf sequencing, RapidPlan for DVH-estimation, FTDC-GPU for optimization dose calculation, AcurosXP-GPU for final dose calculation), and additionally, to support the automated adaptive workflow 220, further includes an additional component that allows for the automatic generation, automatic selection, and automatic continuous modification of optimization parameters 512′ by which the algorithm 511′ and ultimately the generated plan 513′ are optimized based on the clinical goals stored in the 4D directives. Information contained in the 4D directives regarding the treatment parameters of the adapted treatment plan (530, 530A, 530B) are used as the optimization parameters in 512′, and these parameters are automatically modified in 540′ with the aim to obtain a similar dose distribution as the dose distribution of the treatment plan 320′.

When the evaluation module 520′ determines that the dose distribution of the generated plan 513′ is within an accepted threshold, the so generated treatment plan is accepted as the adapted treatment plan 530C. The adapted treatment plan 530C therefore combines the optimization based on the changes in the anatomy of the patient with the optimization based on WSI. Therefore, the adapted treatment plan 530C provides a comprehensively optimized treatment plan.

A treatment plan may also be adapted between different treatment sessions/therapies using the treatment plan adaptation module 510. For example, in a situation where the patient 110 already underwent a radiation therapy treatment and is now to be subjected to a further radiotherapy treatment, a new WSI may be ordered prior to the further radiotherapy treatment to reflect the impact of the radiotherapy treatment on the tumor. For instance, the % of necrotic tissue in the WSI may indicate how susceptible the tumor was towards the radiotherapy treatment. To make sure that the WSI comprises the required information, the WSI may be specifically ordered in terms of staining and pre-processing to best reflect the impact of the previous treatment(s).

The information obtained from the new WSI may be used to further adapt the treatment plan used for the first radiation therapy. For example, the radiation dose may be escalated or de-escalated.

Additionally, or alternatively, a prior WSI may be obtained which was taken before the first radiotherapy treatment. This prior WSI may be compared to the WSI taken after the radiotherapy treatment. The result of the comparison between the two WSIs may also be used to further adapt the radiotherapy treatment plan. In order to be able to correctly compare the two WSIs, the WSI obtained after the second radiotherapy treatment may need to be specifically ordered to be comparable with the prior WSI in terms of staining and/or pre-processing.

An exemplary process S100 for adapting a radiation treatment plan based on the specific characteristics of the target (tumor) is shown in FIGS. 16A, 16B. In a first optional step S101, a treatment plan is generated for a patient based on radiology image data of an anatomy of interest containing the target. Alternatively, instead of generating a treatment plan, in the first step S101, a previously generated treatment plan is received/obtained from a treatment plan generating system/database/server. Then, a whole slide image (WSI) of the target is obtained in S102. The WSI may be obtained from any external digital databases containing the WSI. An adapted treatment plan is next generated in S103 based on information extracted from the WSI. The adapted treatment plan is evaluated in S104 using one or more additional data and/or information, including but not limited to, omics data, EMR data, radiology data, pathology data. Based on the results of the evaluation, a decision is made in S105 as to the next steps. This may involve revising the treatment plan, and or suggesting further exams/studies/alternative treatments, etc.

An exemplary process S200 for adapting a radiation treatment plan based on the specific characteristics of the target (tumor) as well as a change in the anatomy of the patient is shown in FIG. 16C. In a first optional step S201, a treatment plan is generated for a patient based on radiology image data of an anatomy of interest containing the target. Alternatively, instead of generating a treatment plan, in the first step S201, a previously generated treatment plan is received/obtained from a treatment plan generating system 300/database/server. Then, a whole slide image (WSI) of the target is obtained in S202. The WSI may be obtained from any external digital databases containing the WSI. An adapted treatment plan is next generated in S203 based on information extracted from the WSI. In S204, a treatment session image (CBCT) of the anatomy of interest containing the target is generated. The adapted treatment plan generated in S203 is updated in S205 based on information determined from the CBCT image. The information determined from the CBCT image includes information regarding the shift in the anatomy of interest. The updated adapted treatment plan is then evaluated in S206 and a decision is made in S207 regarding further steps to be taken. This may involve revising the treatment plan, and or suggesting further exams/studies/alternative treatments, etc.

An exemplary process S300 for adapting a radiation treatment plan based on information contained in a new WSI taken in between radiation therapies is shown in FIG. 16D. In step S301, a radiation treatment is delivered to the patient according to a radiation treatment plan. Prior to administering a further radiation therapy, a new WSI is obtained for the patient in S302. Based on the information extracted from the new WSI, the treatment plan is updated in S303. The updated treatment plan is evaluated in S304, and a decision is made in S305 regarding further steps to be taken.

An exemplary process S400 for adapting a radiation treatment plan based on a comparison between information contained in a new WSI taken in between radiation therapies and the WSI corresponding to the first radiation therapy is shown in FIG. 16E. In step S401, a radiation treatment is delivered to the patient according to a radiation treatment plan. Prior to administering a further radiation therapy, a new WSI is obtained for the patient in S402. In S403, information extracted from the new WSI is compared with the information contained in the WSI corresponding to the delivered radiation treatment plan. In S404, the treatment plan is updated based on the comparison. The updated treatment plan is evaluated in S405, and a decision is made in S406 regarding further steps to be taken.

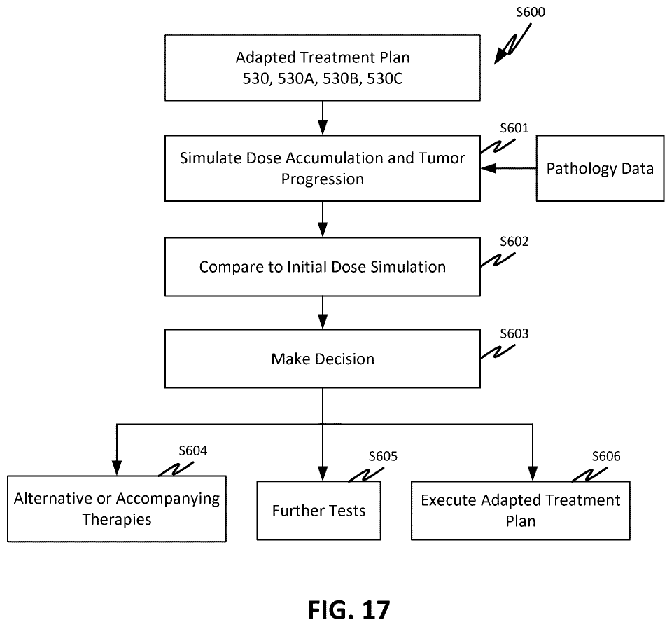

The adapted treatment plans (530, 530A, 530B, 530C, etc.) generated based on the methods described throughout this specification, as well as the associated pathology data stored in the EHR 900 can also be applied to a dose calculation algorithm S601 as shown in FIG. 17 to simulate the dose accumulation in the session target volume and tumor progression according to the adapted treatment plans. The simulated dose and tumor progression can be compared in S602 with the dose simulation and tumor state in the treatment plan 320′. A decision is next made in S603 as to the further course of action based on the result of the comparison. This provides an additional check and validation before radiation delivery.

Depending on the outcome, alternative or accompanying therapies, such as, but not limited to, chemotherapy, immunotherapy, surgery, etc. may be performed in S604, and/or additional exams such as lab tests, sequencing, additional radiology imaging exams, additional digital pathology exams, etc. in S605, or the adapted treatment plan is executed in S606.

The usage of digital pathology data in radiotherapy may be fed back to the digital pathology workflow as follows:
A biopsy and/or the preparation of a WSI may be ordered and scheduled in response to the generation of the treatment plan.
The stains may be automatically specified.
Staining/analysis of neighboring slices may be requested.
The pre-processing required may be specified, e.g., by way of an application of specific image analysis algorithms.

It is thus apparent that the disclosed subject matter enables an adaptive process 220 that integrates radiation therapy workflow with digital pathology workflow in a seamless manner.

It is also apparent that the disclosed subject matter enables a system to perform the adaptive workflow 220 as described herein.

It is thus also apparent that the disclosed subject matter enables the adaptation of radiation treatment plans to the characteristics of the individual tumors as well as anatomical changes in the patient.

It is thus also apparent that the disclosed subject matter enables the adaptation of treatment plans according to the cellular properties of the tumor.

It is also apparent that the disclosed subject matter allows for a more personalized and better targeted radiography protocol that is adapted to the particular cancer type.

It will be appreciated that the aspects of the disclosed subject matter can be implemented, fully or partially, in hardware, hardware programmed by software, software instruction stored on a computer readable medium (e.g., a non-transitory computer readable medium), or any combination of the above.

For example, components of the disclosed subject matter, including components such as a controller, process, or any other feature, can include, but are not limited to, a personal computer or workstation or other such computing system that includes a processor, microprocessor, microcontroller device, or is comprised of control logic including integrated circuits such as, for example, an application specific integrated circuit (ASIC).

Features discussed herein can be performed on a single or distributed processor (single and/or multi-core), by components distributed across multiple computers or systems, or by components co-located in a single processor or system. For example, aspects of the disclosed subject matter can be implemented via a programmed general purpose computer, an integrated circuit device, (e.g., ASIC), a digital signal processor (DSP), an electronic device programmed with microcode (e.g., a microprocessor or microcontroller), a hard-wired electronic or logic circuit, a programmable logic circuit (e.g., programmable logic device (PLD), programmable logic array (PLA), field-programmable gate array (FPGA), programmable array logic (PAL)), software stored on a computer-readable medium or signal, an optical computing device, a networked system of electronic and/or optical devices, a special purpose computing device, a semiconductor chip, a software module or object stored on a computer-readable medium or signal.

When implemented in software, functions may be stored on or transmitted over as one or more instructions or code on a computer-readable medium. The steps of a method or algorithm disclosed herein may be embodied in a processor-executable software module, which may reside on a computer-readable medium. Instructions can be compiled from source code instructions provided in accordance with a programming language. The sequence of programmed instructions and data associated therewith can be stored in a computer-readable medium (e.g., a non-transitory computer readable medium), such as a computer memory or storage device, which can be any suitable memory apparatus, such as, but not limited to read-only memory (ROM), programmable read-only memory (PROM), electrically erasable programmable read-only memory (EEPROM), random-access memory (RAM), flash memory, disk drive, etc.

As used herein, computer-readable media includes both computer storage media and communication media, including any medium that facilitates transfer of a computer program from one place to another. Thus, a storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer.

Also, any connection is properly termed a computer-readable medium. For example, if the software is transmitted from a website, server, or other remote source using a transmission medium (e.g., coaxial cable, fiber optic cable, twisted pair, digital subscriber line (DSL), or wireless technologies such as infrared, radio, and microwave), then the transmission medium is included in the definition of computer-readable medium. Moreover, the operations of a method or algorithm may reside as one of (or any combination of) or a set of codes and/or instructions on a machine readable medium and/or computer-readable medium, which may be incorporated into a computer program product.

One of ordinary skill in the art will readily appreciate that the above description is not exhaustive, and that aspects of the disclosed subject matter may be implemented other than as specifically disclosed above. Indeed, embodiments of the disclosed subject matter can be implemented in hardware and/or software using any known or later developed systems, structures, devices, and/or software by those of ordinary skill in the applicable art from the functional description provided herein.

In this application, unless specifically stated otherwise, the use of the singular includes the plural, and the separate use of “or” and “and” includes the other, i.e., “and/or.” Furthermore, use of the terms “including” or “having,” as well as other forms such as “includes,” “included,” “has,” or “had,” are intended to have the same effect as “comprising” and thus should not be understood as limiting.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

The terms “system,” “device,” and “module” have been used interchangeably herein, and the use of one term in the description of an embodiment does not preclude the application of the other terms to that embodiment or any other embodiment.

Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention.

1. A method for adapting a treatment plan, comprising:
obtaining a treatment plan generated based on radiology image data of an anatomy of interest containing a target;
obtaining a whole slide image (WSI) of the target; and
updating the treatment plan based on information extracted from the WSI.

2. The method of claim 1, wherein the extracted information includes information specific to cellular properties of the target.

3. The method of claim 2, wherein the target is a tumor and the extracted information includes one or more biomarkers.

4. The method of claim 3, wherein the biomarkers include biomarkers that encode morphological information about the tumor, biomarkers that include information about proliferation status or immune status of the tumor, and biomarkers that indicate the tumor's resistance to ionizing radiation.

5. The method of claim 1, wherein the extracted information is presented as an average feature vector.

6. The method of claim 5, further comprising extracting the average feature vector from the WSI by:
applying a trained machine learning system to the WSI to convert WSI image data into feature vectors; and
calculating an average of the feature vectors,
wherein the machine learning system has been trained to predict a WSI-level tissue or cell morphology classification, regression, or resistance.

7. The method of claim 6, wherein the converting includes:
segmenting the WSI into a plurality of tiles/patches; and
assigning a corresponding feature vector to each of the tiles/patches.

8. The method of claim 7, wherein the trained machine learning system includes a trained ResNet 50 deep learning neural network model.

9. The method of claim 5, wherein the updating includes:
using the treatment plan and the WSI as inputs to a plan adaptation module, the plan adaptation module including optimization parameters generated based on the feature vector extracted from the WSI; and
generating an updated treatment plan by optimizing the treatment plan based on the average feature vector.

10. The method of claim 9, wherein the optimization parameters include radiation and/or radiation dose related parameters.

11. The method of claim 10, wherein the optimizing includes modifying the radiation and/or radiation dose related parameters based on information contained in the average feature vector.

12. The method of claim 9, wherein the plan adaptation module is one of a rule-based module and a deep learning based module.

13. The method of claim 12, wherein the deep learning based module is trained to generate treatment plan parameters based on feature vectors.

14. The method of claim 13, wherein the training includes training a neural network model using data sets of a plurality of patients suffering from a specific disease type/subtype and associated WSIs and radiation treatment plans, wherein the trained neural network model is configured to predict treatment plan parameters based on feature vectors.

15. The method of claim 1, wherein the updating is further based on additional data, the additional data including one or more of omics data, EMR data, and one or more additional radiology image data.

16. The method of claim 15, wherein the additional radiology image data includes information regarding a shift in the anatomy of interest containing the target.

17. The method of claim 16, further comprising:
evaluating the updated treatment plan; and
selecting a next course of action based on a result of the evaluation.

18. The method of claim 17, wherein
the evaluating includes simulating radiation dose accumulation and disease progression for the updated treatment plan and comparing it to simulated radiation dose accumulation generated for the treatment plan; and
the selecting includes one or more of revising the treatment plan, ordering new tests, obtaining new WSI, proposing alternative or accompanying therapies, perform additional checks, and executing the updated treatment plan.

19. An automated workflow for an adaptive radiation therapy session, comprising:
obtaining a treatment plan generated for a target within a patient based on radiology image data;
obtaining a whole slide image (WSI) of the target;
extracting a feature vector from the whole slide image (WSI), the feature vector containing cellular information about the target;
updating the treatment plan based on the feature vector;
evaluating the updated treatment plan using additional pathological and radiological information about the target; and
selecting a next course of action based on a result of the evaluation,
wherein the additional pathological information includes information pulled from an EHR database and/or from an EMR of the patient,
wherein the additional radiology information includes information obtained from a CBCT scan of the target, and
wherein the next course of action includes one or more of revising the treatment plan, ordering new tests, obtaining new WSI, proposing alternative or accompanying therapies, perform additional checks, and executing the updated treatment plan.

20. A treatment planning system, comprising:
a computer processing system including a user interface;
a memory device storing software instructions for a treatment plan generating module configured to generate a treatment plan for delivering a prescribed radiation dose to a target based on prescribed clinical goals received via the user interface;
a converting module configured to convert whole slide image (WSI) data into feature vectors, the feature vectors including cellular information about the target; and
a treatment plan adapting module configured to automatically update the treatment plan based on the feature vectors,
wherein the treatment plan adapting module uses the treatment plan and an average of the feature vectors as inputs to a plan adaptation algorithm and generates an updated treatment plan by optimizing the treatment plan based on the average feature vector.

21. A non-transitory computer-readable storage medium having computer instructions stored thereon for automatically updating treatment plans based on whole slide image (WSI), which when executed by a processor, cause the processor to:
convert whole slide image (WSI) data into an average feature vector, the feature vector including cellular information about a target; and
automatically update the treatment plan based on the average feature vector,
wherein the updating includes using the treatment plan and the average vector as input to a trained treatment plan adaptation algorithm and generating an updated treatment plan by optimizing the treatment plan based on the average feature vector.
